(19) United States
(12) Patent Application Publication
(10) Pub. No.: US 2019/0134302 A1
(43) Pub. Date: May 9, 2019

(54) IMPLANTABLE FLUID DELIVERY DEVICE

(71) Applicants: ECOLE POLYTECHNIQUE FEDERALE DE LAUSANNE (EPFL), Lausanne (CH); Antlia SA, Lausanne (CH)

(72) Inventors: Michel BACHMANN, Vaux-sur-Morges (CH); Nikolaos STERGIOPULOS, Préverenges (CH)

(21) Appl. No.: 16/184,577

(22) Filed: Nov. 8, 2018

(30) Foreign Application Priority Data
Nov. 8, 2017 (EP) 17200528

(51) Int. Cl. A61M 5/142; A61M 5/172

(52) U.S. Cl. CPC A61M 5/14276; A61M 5/14232; A61M 2230/201; A61M 2230/04; A61M 2205/3523; A61M 5/1723

(57) ABSTRACT

An implantable delivery device for delivering a fluid in a controlled manner, including a body having a chamber, a tube having an inlet and an outlet, at least a portion of the tube passing through the chamber, a drive pinion operatively connected to a motor for rotation, a wheel engaging with the drive pinion, and a ring operatively attached to the wheel, wherein the ring presses against the tube in a pressing zone and is driven by a rotation of the wheel relative to the body such that the pressing zone moves along the tube resulting from the rotation of the wheel to generate a movement of fluid present in the tube from the inlet to the outlet.

### CROSS-REFERENCE TO RELATED APPLICATIONS

The present patent application claims foreign priority to European Patent Application with the Serial No. EP 17200528 filed on Nov. 8, 2017, the entire contents of this reference herewith incorporated by reference in its entirety.

### FIELD OF THE INVENTION

The present invention generally relates to an implantable device. More specifically, the present invention relates to a high-precision implantable fluid delivery device with reservoir means for example for delivering fluid to a living body (such as a human or an animal). A typical application is in the field of insulin pumps used to treat diabetes.

### BACKGROUND

Implantable medical devices suitable for the delivery of drugs are known per se in the art. Swiss patent No 688 224 discloses an implantable device for the delivery of liquid pharmaceutical drugs in the human body. Other examples of similar devices are described in U.S. Pat. Pub. No. 2004/0249365, U.S. Pat. Nos. 7,981,107, 7,201,746, Int. Pat. Pub. No. WO 2002/083207, German Patent No. DE 4123091, Int. Pat. App. No. PCT/US2003/012367, UK Patent No. GB 2174218 and Int. Pat. App. No. PCT/US1996/009608, all incorporated by reference in their entirety in the present application.

In the above-mentioned Swiss patent No 688 224, the disclosed device comprises an axial piston pump. The piston is driven under control in rotation and axial translation. A fluid reservoir is connected to a suction side of the pump. The pump preferably has a ceramic cylinder and piston. A refilling connection for the reservoir is re-sealable. The integral rotary drive has a separate control unit. Pump, reservoir and drive are coaxial in a cylindrical casing. Alternatively the drive is external, a non-contact coupling transmitting rotary motion to the piston. The end face of the cylinder has a cam profile. An eccentric cam follower peg, which produces the axial motion, is attached to the piston. This system is rather complicated as it involves at least two displacements of the piston, i.e. a rotation coupled to a translation. According to this geometry, it is necessary to use specific means to transform the rotation created by the motor into a translation. The disclosed means complicate the construction, are a dysfunction risk and consume energy.

Another prior art pump is known from U.S. Patent Application No. 2004/0044332. This publication discloses an implantable device for delivering medicines in liquid form comprising: a reservoir provided with an inlet and an outlet, said reservoir being adapted to expel the liquid; a variable volume chamber provided with an inlet and an outlet, the volume of the variable volume chamber being in particular smaller than that of the reservoir; a first conduit communicating the outlet of the reservoir with the inlet of the variable volume chamber to fill the latter; a second conduit whereof one of the ends is connected on the outlet of the variable volume chamber.

In this prior art, in fact two variable volume chambers are used (one being the reservoir), separated by a valve, and function by using their respective restoring forces to expel a desired quantity of liquid, for example medicine.

A further prior art fluid delivery device is described in WO 2008/020272. This implantable delivery device comprises at least a body, an inlet and an outlet and actuating means. The actuating means comprise at least an actuator displacing a piston in a rotating cylinder forming at least one variable volume chamber, the volume of which is varied by rotation of said cylinder thereby pumping liquid such as a drug from a reservoir through said inlet and expelling said pumped liquid from said variable volume chamber through at least a first temporary chamber and said outlet by variation of said volume.

However, despite all these features of the state of the art implantable fluid delivery devices, there is still a strong need to provide for novel and strongly improved fluid delivery devices, having a small size, improved reliability, and permitting very fine dosages.

### SUMMARY

According to one aspect of the present invention, the proposed implantable fluid delivery device has the goal to improve the known devices and methods, by providing for a simple device with reduced complexity that is more reliable.

According to another aspect of the present invention, a system or device is provided for fluid delivery that allows a fine dosage of the expelled liquid.

According to still another aspect of the present invention, a system or device is provided that is small in size allowing small animal research and laparoscopic implantation.

Moreover, according to yet another aspect of the present invention, a system or device is provided that can provide for a reservoir suitable for use with the present pump.

According to one aspect of the present invention, an implantable delivery device for delivering a fluid in a controlled manner is provided. Preferably, the device includes a body having a chamber, a tube having an inlet and an outlet, at least a portion of the tube passing through the chamber, a drive pinion operatively connected to a motor for rotation, a wheel engaging with the drive pinion, and a ring operatively attached to the wheel, wherein the ring presses against the tube in a pressing zone and is driven by a rotation of the wheel relative to the body such that the pressing zone moves along the tube resulting from the rotation of the wheel to generate a movement of fluid present in the tube from the inlet to the outlet.

According to still another aspect of the present invention, a peristaltic pumping mechanism is provided for delivering a fluid. Preferably, the peristaltic pumping mechanism preferably includes a body having a circular peristaltic chamber, a flexible tube having an inlet and an outlet, the tube arranged at a circular side wall of the circular peristaltic chamber, an axis arranged through a rotational center of the circular peristaltic chamber, a sleeve bearing arranged around the axis in an area of the circular peristaltic chamber, a disk rotatably attached to the body via the axis, at least two peristaltic rollers urging against the flexible tube towards the circular side wall inside the circular peristaltic chamber. Moreover, preferably, the disk and the at least two peristaltic rollers are configured such that while the disk rotates around the axis relative to the body, the at least two peristaltic rollers are driven by the disk to rotate around the axis along a circular orbit and also rotate around their own rotational axis for performing a peristaltic movement to the tube, and the at least two rollers and the sleeve bearing are in contact with each other to couple a rotation of one of the rollers to the other one of the rollers.

According to another aspect of the present invention, a peristaltic pump is provided that is made of a reduced number of parts as compared to background art peristaltic pumps, by coupling a rotation of two or more peristaltic rollers via a central axis, and providing an enclosure of the circular peristaltic chamber for the peristaltic movement with a wheel that has a dual purpose of rotatively driving the peristaltic rollers and providing for a cover of the circular peristaltic chamber.

According to some aspects of the invention, the resulting device or system is a promising solution, in which the idea is to infuse fluids, such as specific drugs, directly into a body location using a high-precision, telemetrically controlled, implantable drug infusion device. A high-concentration drug can be stored within the implant and the dosing is low so that drug delivery can take place over long periods, for example but not limited to weeks to months or years. Alternative designs may incorporate access ports for transcutaneous drug refill. Because the drugs are administered locally with non-systemic effects, the technology probes for a user-defined, low-dosage, high-precision and high efficiency drug delivery system.

Other advantages of the present invention include for example but are not limited to the following features:

Small compact size to facilitate peripheral implantation, intracranial implantation, cardiac applications, laparoscopic insertion and small animal research;

High-precision bolus administration;

Dosing independent of load, for example but not limited to arterial pressure or intracranial pressure at distal end of delivery catheter;

Externally programmable/activated for continuous, periodic or user-defined drug administration; and

Magnetic resonance imaging (MRI) safe and compatible.

The above and other objects, features and advantages of the present invention and the manner of realizing them will become more apparent, and the invention itself will best be understood from a study of the following description with reference to the attached drawings showing some preferred embodiments of the invention.

### BRIEF DESCRIPTION OF THE SEVERAL VIEWS OF THE DRAWINGS

Other features and advantages of the present invention will appear more clearly from reading the following detailed description of embodiments of the invention which are presented solely by way of non-restrictive examples and illustrated by the attached drawings in which:

FIG. 1 illustrates a perspective view of an embodiment of the invention;

FIG. 2 illustrates a partial cut view of an embodiment of the invention;

FIG. 3 illustrates a cut view along A-A of an embodiment of the invention;

FIG. 4 illustrates a cut view along B-B of an embodiment of the invention; and

FIGS. 5A and 5B illustrate an embodiment of the present invention and a detail thereof.

Herein, identical reference numerals are used, where possible, to designate identical elements that are common to the figures. Also, the images are simplified for illustration purposes and may not be depicted to scale.

### DETAILED DESCRIPTION OF THE EMBODIMENTS

In the following description, the same references will be used to identify the same or corresponding elements in the different figures of the present application for the sake of clarity and simplicity.

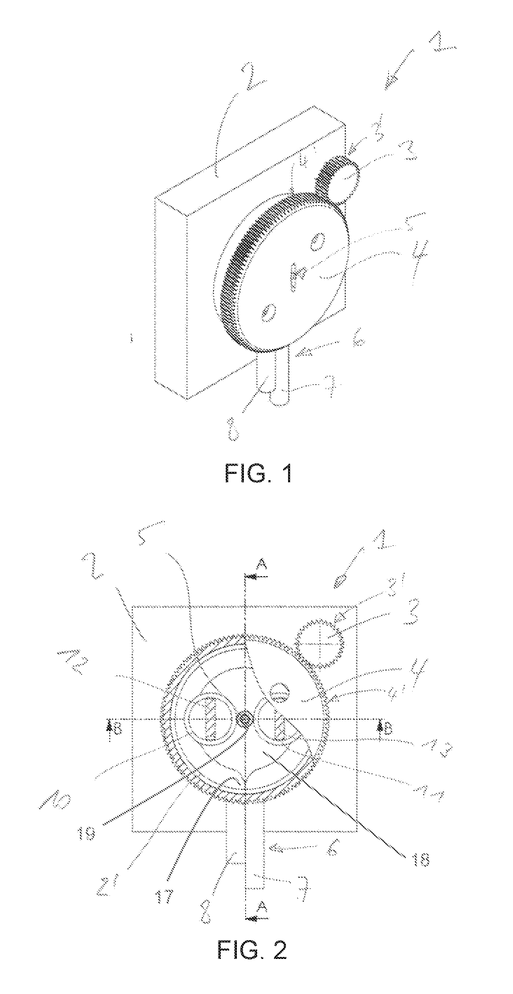

FIG. 1 illustrates a perspective schematic view of the pumping unit or device, being a peristaltic pump, or peristaltic pumping mechanism, according to an embodiment of the invention. The device or unit 1 thus comprises a body 2 with a drive pinion 3 driven by a motor 16, shown exemplarily and schematically in FIG. 4. As shown in this exemplary embodiment, body 2 has a circular chamber 18, and is preferably made of one piece to provide for rigidity to the circular chamber 18. Also, drive pinion 3 is mounted to a peripheral portion of body 2.

The pinion or dented wheel 3 is meshed with an actuating wheel, disk, or drum 4 via teeth 3′ of pinion 3 and a ledge 4′ of wheel 4 that rotates around an center axis 5. A flexible tube 6 having an inlet 7 and an outlet 8 passes in the unit 1 as will be described in more detail below. Axis 5 is also mounted to a central portion of body 2.

FIG. 2 illustrates a front partially cut view of an embodiment of the present invention of the device shown in FIG. 1. The partial cut of wheel 4, the cut view provided for illustration and explanary purposes, allows to understand the functioning of the pumping unit 1. As shown, the unit 1 further comprises two rings, rollers, drums 10, 11 that press on the tube 6 on one side and against axis 5 on the other side of the rings 10, 11. In addition, two extensions 12, 13 extend in the rings 10, 11. The extensions 12, 13 are parts that are attached to the wheel 4. They may be fixed to the wheel or integral with the wheel 4 such that the wheel and extensions are one single part. As the wheel 4 and the extension 12, 13 are linked to each other, the extensions 12, 13 rotate with the wheel 4, when wheel 4 is driven by pinion 3 through teeth 3′ and ledge 4′, via motor 16.

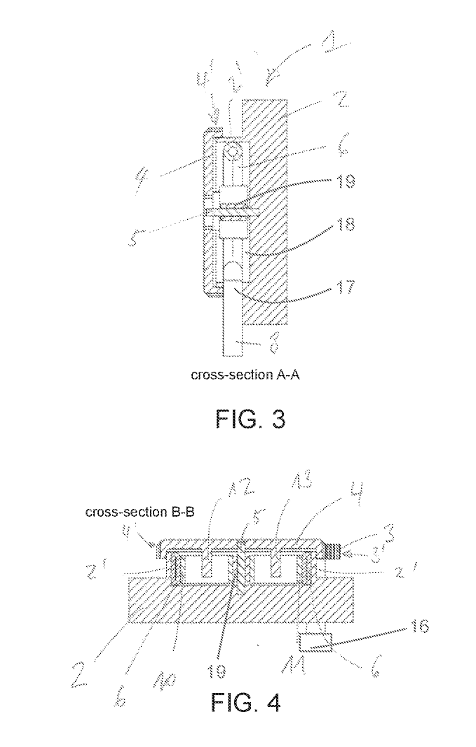

As shown in FIGS. 2 and 3, wall 2′ of unit 2 are formed as a circular structure, forming a circular opening in the center of unit 2 forming the peristaltic chamber 18, and flexible tube 6 is arranged around the circular structure of the inner circular face of wall 2′ to form a loop inside the circular opening, with inlet 7 and outlet 8 of tube 6 traversing the wall 2′ via an opening 17 in wall 2′. Preferably, wall 2′ and chamber 18 have a circular shape. In the variant shown, the wall 2′ has one opening for both inlet 7 and outlet 8 of tube 6, such that inlet 7 and outlet 8 are touching each other along a side. Inlet 7 and outlet 8 are arranged in parallel to each other to exit opening 17 of chamber 18, as shown in FIGS. 1-2. For example, tube 6 can be made from a medical-grade elastomeric tubing.

The rotation of extensions 12, 13 allows to drag the rings or rollers 10, 11 that will in fact rotate on themselves and around axis 5 in a circular orbit as they are pressing on the tube 6 against the circular inner face 2′ of wall 2′, due to a rotation performed by wheel 4 via motor 16, relative to unit 2. With this movement, the rings 10 or rollers 11 will roll like a wheel on tube 6, and compress tube 6 against inner surface of wall 2′, due to the rotation of rings or rollers 10, 11 around their own rotational axis, in addition to the circular orbit around axis 5. This will prevent any sliding movement of rings or rollers 10, 11 relative to surface of tube 6, and will substantially reduce wear and abrasion to tube 6. As the tube 6 is compressed or squeezed against a wall 2′ of the unit 2, see in FIG. 4 for example, this movement of the rings 12, 13 pressing on the tube 6 that is squeezed on the wall 2′ will realize a drive effect in the tube 6 that will in turn allow liquid or fluid in the tube 6 to enter the pump at the inlet 7 and exit the unit 1 at the outlet 8 thereby realizing a pumping effect with the rotating wheel 4, to perform a peristaltic movement of a liquid or fluid inside flexible tube 6.

Moreover, as shown in FIGS. 2-4, the rollers or rings 10, 11 rotated in a circular orbit or trajectory around axis 5 by the extensions 12, 13, respectively. In addition, rollers or rings 10, 11, are also configured to rotate around their own rotational center axis. Moreover, for both rollers or rings 10, 11, their outer surface, for example a cylindrical surface, is also in contact with a hollow cylinder, cylindrical washer, sleeve bearing or bushing 19 that is formed around central axis 5. Sleeve bearing 19 can rotate around axis 5, and can be made of a hard material providing for low friction with respect to axis 5, and can be implemented as a bearing washer with rolling cylinders, axial needle bearing washer, plain bearing, sliding bearing, cylindrical friction bearing. This allows to couple the rotation of roller or ring 10 to the rotation of roller or ring 11, not only by the rotation provided by extensions 12, 13, but also by to rotating cylindrical sleeve bearing 19. For example, while rollers 10, 11 will rotate in a clockwise direction, sleeve bearing 19 that is contact with both rollers 10, 11 on opposite sides, will rotate counter clock-wise, and vice-versa. This arrangement of rings or rollers 10, 11 that abut against sleeve bearing 19 allows to provide for mechanical stability and durability, similar to a roller bearing. Sleeve bearing 19 and axis 5 will act as a counterforce against a force created by the peristaltic motion on rings or rollers 10, 11, and will reduce bending, mechanical wear, and the coupling of the rotation of rollers 10, 11 will also reduce a torque that needs to be applied for performing the rotation by wheel 4 via drive pinion 3 and motor 16.

Therefore, the functioning of the pumping unit 1 and its delivery is regulated by the rotational movement of the wheel 4 driven by the pinion 3, and by the rotating motor 16 acting on the pinion 3 and wheel 4, the rotation performed relative to unit 2.

It is thus easy to regulate the delivery of the fluid or liquid, for example a drug, with this system by driving the rings into rotation with the wheel 4.

Although two rings 10, 11 have been illustrated, it is possible to use more than two rings, for example three or more, in other embodiments and variants of the present invention. In a variant, in lieu of extensions 12, 13, rollers or rings 10, 11 can be made to rotate around themselves relative to wheel 4 by using an axis and a ball bearing rotatably attaching to wheel 4, so as to reduce a friction when engaging with tube 6, as rollers or rings 10, 11 will rotate on tube 6.

FIG. 3 illustrates a cut view of the unit 1 according to an embodiment along axis A-A of FIG. 2 with the same parts identified with the same references. As shown in this cross-sectional view, wheel 4 is formed for the dual purpose of serving as a rotative drive element to rotate the rollers or rings 10, 11, but also as a casing for enclosing the circular chamber 18 that is formed by unit 2, for example by the protruding circular ledge 4′ that is arranged around the circumference of the wheel 4, and in an installed position, overlaps with the outer surface of circular wall 2′. This way, wheel or disk 4 is used to drive rollers or rings 10, 11, but also serves as a protective lid or cover to chamber 18 to protect the peristaltic mechanism. A diameter of the circle formed by wheel or disk 4 is larger than a diameter of the circular chamber 18. A small gap is formed between ledge 4′ of wheel 4 and wall 2′ on unit 2, that circumferentially goes around wall 2′. This gap could also be sealed, for example with another cylindrical sleeve bearing or similar structure providing for reduced friction, or over type of circular sealing. Also, as shown in FIG. 3, a length of ledge 4′ in a direction of axis 5 is such that it does not obstruct the opening 17 for tube 6, with inlet and outlet 7, 8.

FIG. 4 illustrates a cut view of the unit 1 according to an embodiment along axis B-B of FIG. 2 with the same parts identified with the same references. Here, in this figure, one sees the extensions 12, 13 as being parts of the wheel 4 and the rings 10, 11 that press the tube 6 on both sides of the pumping unit body 2. Extensions 12, 13 are elements, for example bolts or rods, that protrude perpendicularly from the surface defined by wheel 4, parallel to an axis of extension of the main rotational axis 5.

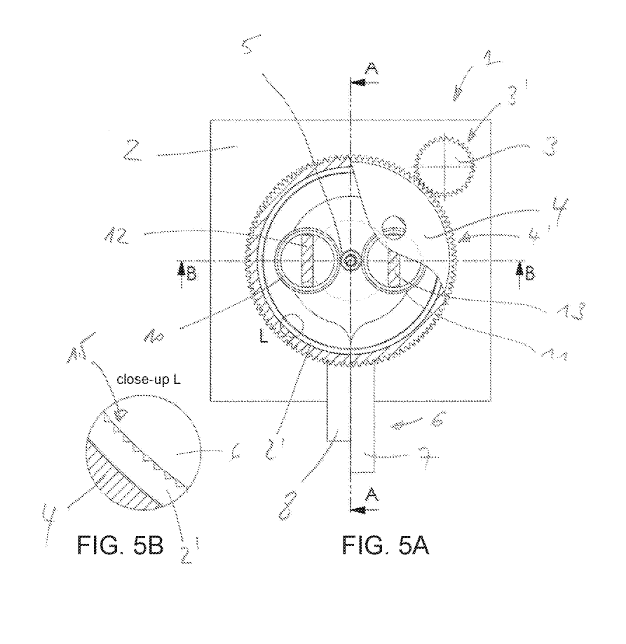

FIGS. 5A and 5B illustrate an embodiment of the present invention in which FIG. 5B shows a detail of FIG. 5A at the area L. In this embodiment, the elements identical or similar to embodiments described above will be identified with the same references. Accordingly, the description made above applies correspondingly to this embodiment.

Here, the pump comprises again a unit 1 with the same elements as for example illustrated in FIGS. 1-4 and discussed above. In this embodiment, the wall 2′ of the unit 2 comprises additional means such that the tube is maintained in position although the rotation of the wheel 4 and of the rings 10, 11 tends to move the tube 6. These means are in the shape of small teeth 15 which are placed on the inner side of wall 2′. The teeth 15 may be present on the entire side of the wall 2′ or only on parts of it. Also, the teeth 15 may have different sizes or shapes and the means are not limited to teeth as illustrated but other equivalent means are possible.

The device 1 according to some aspects of the present invention may be included in an implantable pump with a reservoir and electronic means, such as but not limited to a chip, electronic circuits, and electric supply such as a battery, for forming an autonomous and programmable device that is able to deliver a chosen quantity of fluid or liquid, such as a drug, at defined moments or upon request.

The pump, in the embodiments described above, may comprise a telemetry system, such as an radio-frequency identification device (RFID) or Bluetooth module which would allow the pump to receive and also send signals and information to an external control unit. The external control unit may be used to program the implant function, for example but not limited drug release rates, drug dosage, time profile of bolus delivery, but it can also be used to collect functional data from the implanted device, such as history of doses, battery status, state of the good functioning of the device etc. The telemetry system may be also used to deliver energy to the implant, in a manner similar to other telemetrically energized implantable devices, for example such as those described in U.S. Pat. No. 5,820,589, this reference herewith incorporated by reference in its entirety.

Further, the pump in all the embodiments described above, can be connected either physically or wirelessly to a physiological signal sensing system which would provide information as to the state of certain physiological variables, for example but not limited to pressure, temperature, electrical activity. The signal can then be interpreted by the pump, for example by its calculating means, including an appropriate program, and used to decide, for example based on imbedded decision algorithms, whether or not to deliver a therapeutic dose. One such concrete example would be the connection of a pump to a system measuring electrocardiography (ECG) or other brain activity signals, which then would allow the pump to deliver, when needed, for example based on predetermined parameters, drugs intracranially. Another example would be the use of the pump in heart failure patients where end diastolic pressure or volume is measured and this signal is used by the pump to deliver in the vena cava, systemically or directly to the heart cavities inotropic or other appropriate drug function regulating drugs. In a similar fashion, the pump can be coupled to an ECG measuring system and use this signals to decide when to deliver cardiac rhythm management drugs either in the vena cava or directly in the heart muscle or heart cavities.

For small animal applications, for intracranial, peripheral or other human applications it is essential that the pump is compact with a minimal weight and volume. To further reduce weight and volume, it is also possible to remove batteries and to store energy in the implant during drug filling, by elastic deformation of a spring or membrane attached to the reservoir. This energy can be gradually released during emptying of the reservoir and be used accordingly to operate the motor. Energy storage can be also achieved by the natural human or animal motion, used a system similar to the automatic (battery-free) watches.

As can be readily understood from the above description, the system is preferably implanted through a trocar or similar device. Hence, the size (diameter) should not exceed certain values corresponding to the sizes of such devices. Typically, a cross-sectional diameter could be of about 18 mm, but other larger or smaller sizes may be envisaged depending on the application and the location where the device is to be implanted.

The embodiments described in the present application are for illustrative purposes and should not be construed in a limiting manner. Other constructions and embodiments are possible using equivalent means remaining within the scope of the present invention.

Also embodiments may be combined together according to circumstances.

One additional degree of freedom is also given by the relative size (i.e. diameter) of the pinion 3 and the wheel 4 that may be chosen appropriately for the desired application and delivery. Also the engagement between pinion 3 and wheel 4 may be a direct one or via a gear train.

The materials forming the present unit/invention may be any suitable material (metal, synthetic, a mix thereof) that is appropriate for the intended use (for example biocompatible materials in case of an implantation in a living body).

As an example of application, the pump disclosed herein may be used in medical applications (for humans or animals). An example would be an insulin pump for the treatment of diabetes.

For example, in a recent publication e “Intensive Intravenous Infusion of Insulin in Diabetic Cats”, M. Hafner et al., J. Vet. Intern. Med. 2014, Vol. 28, pp. 1753-1759, this reference herewith incorporated by reference in its entirety, the study of the publication focused on the intensive treatment of diabetic cats that showed an improved remission under such intensive treatment. Hence, the pump according to the present invention could typically be used for such a treatment of diabetic cats, and other mammals, showing a similar response to intensive treatments as discussed in this publication). In such an application, the present pump may be combined with a glucose sensor and the sensor could monitor the level of glucose and thus act on the functioning of the pump. Of course, other application in the medical field (or not) are possible and might be envisaged.

Exemplary embodiments have been described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present invention is defined not solely by the claims. The features illustrated or described in connection with one exemplary embodiment may be combined with the features of other embodiments. Such modifications and variations are intended to be included within the scope of the present invention. A number of problems with conventional methods and systems are noted herein and the methods and systems disclosed herein may address one or more of these problems. By describing these problems, no admission as to their knowledge in the art is intended. A person having ordinary skill in the art will appreciate that, although certain methods and systems are described herein, the scope of the present invention is not so limited.

Moreover, while this invention has been described in conjunction with a number of embodiments, it is evident that many alternatives, modifications and variations would be or are apparent to those of ordinary skill in the applicable arts. Accordingly, it is intended to embrace all such alternatives, modifications, equivalents and variations that are within the spirit and scope of this invention, and be given the broadest reasonable interpretation in accordance with the language of the appended claims.

1. An implantable delivery device for delivering a fluid in a controlled manner, comprising:
a body having a circular chamber;
a tube having an inlet and an outlet, at least a portion of the tube passing through the chamber;
a drive pinion operatively connected to a motor for rotation;
a wheel engaging with the drive pinion; and
a first roller operatively engage by the wheel,
wherein the first roller presses against the tube to form a pressing zone and is driven by a rotation of the wheel relative to the body such that the pressing zone moves along the tube resulting from the rotation of the wheel to generate a movement of fluid present in the tube from the inlet to the outlet.

2. The implantable delivery device as claimed in claim 1, further comprising:
a first extension connected to the wheel, configured to rotatively drive the first roller.

3. The implantable delivery device as claimed in claim 2, further comprising:
a second roller; and
a second extension connected to the wheel, configure to rotatively drive the second roller.

4. A pump having the implantable delivery device as claimed in claim 1.

5. The pump as claimed in claim 4, wherein the pump is an implantable pump for implantation in a living body.

6. The implantable delivery device as claimed in claim 3, further comprising:
a center axis allowing for a rotation of the wheel relative to the body;
a sleeve bearing arranged around the axis, configured to rotate relative to the axis,
wherein both outer surfaces of the first and the second roller are in contact with an outer surface of the sleeve bearing.

7. The implantable delivery device as claimed in claim 1, wherein the wheel includes a circular ledge, and the body includes a circular wall arranged around the chamber, such that the circular ledge and the circular wall are overlapping to enclose the chamber.

8. A peristaltic pumping mechanism for delivering a fluid comprising:
a body having a circular peristaltic chamber;
a flexible tube having an inlet and an outlet, the tube arranged at a circular side wall of the circular peristaltic chamber;
an axis arranged through a rotational center of the circular peristaltic chamber;
a sleeve bearing arranged around the axis in an area of the circular peristaltic chamber;
a disk rotatably attached to the body via the axis;
at least two peristaltic rollers urging against the flexible tube towards the circular side wall inside the circular peristaltic chamber,
wherein the disk and the at least two peristaltic rollers are configured such that while the disk rotates around the axis relative to the body, the at least two peristaltic rollers are driven by the disk to rotate around the axis along a circular orbit and also rotate around their own rotational axis for performing a peristaltic movement to the tube, and
wherein the at least two rollers and the sleeve bearing are in contact with each other to couple a rotation of one of the rollers to the other one of the rollers.

9. The peristaltic pumping mechanism as claimed in claim 8, wherein the disk includes a circular ledge, and the body includes a circular wall arranged to surround the circular peristaltic chamber, such that the circular ledge and the circular wall are overlapping to enclose the circular peristaltic chamber.

10. The peristaltic pumping mechanism as claimed in claim 8, wherein a diameter of the disk exceeds a diameter of the circular peristaltic chamber.

11. A method for treating diabetes for performing an intensive, short-term insulin treatment to achieve self-remission from diabetes or a substantial improvement in a diabetic state of disease in a mammal using a insulin pump having the peristaltic mechanism as recited in claim 8.
